(19) United States
(12) Patent Application Publication
(10) Pub. No.: US 2022/0257636 A1
(43) Pub. Date: Aug. 18, 2022

(54) REDUCTION OF BONE RESORPTION, ESPECIALLY IN CHRONIC JOINT DISEASES

(71) Applicant: TECHNISCHE UNIVERSITÄT DARMSTADT, Darmstadt (DE)

(72) Inventors: Meike Julia SAUL, Lorsch (DE); Anett Birgit HEGEWALD, Heppenheim (DE)

(21) Appl. No.: 17/628,674

(22) PCT Filed: Aug. 17, 2020
(86) PCT No.: PCT/EP2020/073023

§ 371 Date: Jan. 20, 2022

(30) Foreign Application Priority Data
Aug. 15, 2019 (DE) 10 2019 122 014.9

(51) Int. Cl. A61K 31/7115; A61P 19/00; C12N 15/113; A61K 45/06; A61K 38/20; A61K 47/64

(52) U.S. Cl. CPC A61K 31/7115; A61P 19/00; C12N 15/113; A61K 45/06; G01N 2800/102; A61K 47/6455; C12N 2310/113; G01N 2333/51; A61K 38/20

(57) ABSTRACT

A TLR7/8 inhibitor for reduction of bone resorption, especially in chronic joint diseases, and to a pharmaceutical composition including the inhibitor. A method for predicting the severity of the course of disease of rheumatoid arthritis in a patient.

### INCORPORATED BY REFERENCE

The Sequence Listing under document FUCHSRPCTUS-ST25.txt, created 01/20/2022 with 7000 bytes is incorporated by reference.

### FIELD OF THE INVENTION

The present invention relates to a TLR7/8 inhibitor for the reduction of bone resorption, in particularly in the case of chronic joint diseases, as well as a pharmaceutical composition comprising the inhibitor. The invention also relates to a method for the prognosis of the severity of the course of the disease rheumatoid arthritis of a patient.

### BACKGROUND OF THE INVENTION

Bone resorption is mediated by osteoclasts and plays an important role in different diseases. In particularly in connection with joint diseases a negative influence of bone resorption has been described. This concerns acute joint diseases such as the arthritis, but in particularly also chronic joint diseases such as the rheumatoid arthritis (RA) and the arthrosis. Also in the case of other diseases such as for example the periodontitis or in the case of implants the engraftment of which is poor or not existent excessive bone resorption plays an important role.

Chronic joint diseases (arthropathies) are the most common chronic diseases of older individuals in Germany. The pathology of arthropathies is complex and multifactorial, but the pathogenesis generally involves an inflammation of the joints which is accompanied by bone resorption and leads to chronic pains and physical impairments. Two of the most common kinds of arthropathies are the rheumatoid arthritis and the arthrosis.

Rheumatoid arthritis is characterized by infiltration of immune cells into the synovial fluid which results in local inflammation and bone resorption. Arthrosis is caused by daily attrition of the joints or by traumatic damages. Older people are therefore most frequently affected. Half of all women and one third of all men develop an arthrosis after the age of 60. Currently, there is no curative treatment for any of these diseases. Today's therapies solely aim at relieving pain and maintaining the mobility of the patients.

### SUMMARY OF THE INVENTION

Therefore, the object of the present invention is the provision of substances for the curative treatment of diseases which are accompanied by an excessive bone resorption. Such diseases are in particularly acute or chronic joint diseases as well as periodontitis and disorders of the engraftment of implants. In particularly, such diseases are also bone metastasis and psoriatic arthritis. It is in particularly an object of the present invention to reduce the bone resorption which comes along with the diseases.

The object is solved by the subject matter of the patent claims.

The object is in particularly solved by a TLR7/8 inhibitor for use for the reduction of bone resorption in the case of diseases which are accompanied by an excessive bone resorption, in particularly in the case of diseases which are selected from the group consisting of chronic joint diseases, acute joint diseases and periodontitis, and/or in the case of disorders of the engraftment of implants. The object is also solved by a TLR7/8 inhibitor for use for the reduction of bone resorption in the case of diseases which are accompanied by an excessive bone resorption, in particularly in the case of diseases which are selected from the group consisting of bone metastasis and psoriatic arthritis.

TLRs (toll-like receptors) form a family of receptors which belong to the group of the type I transmembrane glycoproteins. Till today, 13 TLRs have been identified, and from them the TLRs 1 to 10 are expressed in human beings. Of particular interest are TLR7 and TLR8 which due to their similarities are generally addressed together as TLR7/8. TLR7/8 are endosomal receptors, in particularly for single stranded RNA (ssRNA).

Now, surprisingly, it has been found that TLR7/8 essentially contributes to osteoclast differentiation and thus to bone resorption, in particularly in the case of chronic joint diseases so that an inhibition of TLR7/8 can be used for the reduction of bone resorption. In particularly, according to the present invention, it is possible to inhibit TLR7/8 directly with respective direct inhibitors such as for example DN 2087, or indirectly, for example by an inhibition of miR-574-5p.

mIR-574-5p belongs to the class of the so-called microRNAs (miRs), a class of small, non-coding RNAs with a function in gen regulation. Extracellular miRs are secreted in so-called small extracellular vesicles (sEVs) into human body fluids such as the synovial fluid so that they are protected from degradation by ribonucleases (Cheng L., Sharples R. A., Scicluna B. J. and Hill A. F. (2014). “Exosomes provide a protective and enriched source of miRNA for biomarker profiling compared to intracellular and cell-free blood.” J Extracell Vesicles 3).

Functionally, two fundamentally different main modes of action of miRs can be distinguished. On the one hand, miRs bind to their target mRNA in a sequence-dependent manner and result in their translational repression or degradation (Ambros V. (2004). “The functions of animal microRNAs.” Nature 431: 350-355; Lytle J. R., Yario T. A. and Steitz J. A. (2007). “Target mRNAs are repressed as efficiently by microRNA-binding sites in the 5′ UTR as in the 3′ UTR.” Proceedings of the National Academy of Sciences 104: 9667-9672). But, on the other hand, miRs can also activate the gene expression by binding to RNA binding proteins and inhibiting their functions (Eiring A. M., Harb J. G., Neviani P., Garton C., Oaks J. J., Spizzo R., Liu S., Schwind S., Santhanam R., Hickey C. J., Becker H., Chandler J. C., Andino R., Cortes J., Hokland P., Huettner C. S., Bhatia R., Roy D. C., Liebhaber S. A., Caligiuri M. A., Marcucci G., Garzon R., Croce C. M., Calin G. A. and Perrotti D. (2010). “miR-328 functions as an RNA decoy to modulate hnRNP E2 regulation of mRNA translation in leukemic blasts.” Cell 140(5): 652-665; Saul M. J., Baumann I., Bruno A., Emmerich A. C., Wellstein J., Ottinger S. M., Contursi A., Dovizio M., Donnini S., Tacconelli S., Raouf J., ldborg H., Stein S., Korotkova M., Savai R., Terzuoli E., Sala G., Seeger W., Jakobsson P. J., Patrignani P., Suess B. and Steinhilber D. (2019). “miR-574-5p as RNA decoy for CUGBP1 stimulates human lung tumor growth by mPGES-1 induction.” Faseb j: fj201802547R.). Recently, the activation of TLR7/8 (toll-like receptor 7/8) has been described as an alternative mode of action of the two miRs miR-29b and miR-21 in connection with lung cancer (Fabbri M., Paone A., Calore F., Galli R., Gaudio E., Santhanam R., Lovat F., Fadda P., Mao C., Nuovo G. J., Zanesi N., Crawford M., Ozer G. H., Wernicke D., Alder H., Caligiuri M. A., Nana-Sinkam P., Perrotti D. and Croce C. M. (2012). “MicroRNAs bind to Toll-like receptors to induce prometastatic inflammatory response.” Proc Natl Acad Sci USA 109: E2110-2116; Salvi V., Sozzani S., Bosisio D., Salvi V., Gianello V., Busatto S., Bergese P., Andreoli L., Oro U., Zingoni A., Tincani A., Sozzani S. and Bosisio D. (2018). “Exosome-delivered microRNAs promote IFN-a secretion by human plasmacytoid DCs via TLR7.” JCI Insight 3). Also miR-574-5p has already been identified as a TLR7/8 ligand (WO 2017/079983 A1a.

However, a role of miRs in bone resorption in the case of chronic joint diseases has not been known up to now. In particularly, it has also not been known that EV mediated miRs can act in a similar manner like a hormone.

In the present case it has been found that miR-574-5p plays a decisive role in connection with bone resorption, in particularly in the case of chronic joint diseases. miR574-5p from sEVs from the synovial fluid of patients with rheumatoid arthritis leads via an activation of TLR7/8 to an increased osteoclast differentiation, such as explained in detail in the section of examples. This is in particularly also surprising due to the fact that up to now it has been assumed that TLR7/8 agonists inhibit the osteoclast differentiation (Miyamoto A. et al. (2012) R848, a toll-like receptor 7 agonist, inhibits osteoclast differentiation but not survival or bone-resorbing function of mature osteoclasts).

Thus, the present invention relates to a TLR7/8 inhibitor for use for the reduction of bone resorption in the case of diseases which are accompanied by an excessive bone resorption, in particularly in the case of diseases which are selected from the group consisting of chronic joint diseases, acute joint diseases and periodontitis, and/or in the case of disorders of the engraftment of implants. Preferred are TLR7/8 inhibitors for use for the reduction of bone resorption in the case of chronic joint diseases. The invention also relates to a TLR7/8 inhibitor for use for the reduction of bone resorption in the case of diseases which are accompanied by an excessive bone resorption, in particularly in the case of diseases which are selected from the group consisting of bone metastasis and psoriatic arthritis.

The TLR7/8 inhibitors of the present invention reduce the bone resorption, in particularly by means of an inhibition of the osteoclast genesis and/or the osteoclast differentiation, preferably in the case of diseases from the group consisting of chronic joint diseases, acute joint diseases and periodontitis, and/or in the case of disorders of the engraftment of implants. A preferred acute joint disease is the arthritis. The TLR7/8 inhibitors of the present invention reduce the bone resorption, in particularly by means of an inhibition of the osteoclast genesis and/or the osteoclast differentiation, preferably also in the case of diseases from the group consisting of bone metastasis and psoriatic arthritis.

The chronic joint disease is preferable selected from the group consisting of rheumatoid arthritis and arthrosis. Particularly preferably, the chronic disease is rheumatoid arthritis.

According to the present invention, inhibitors of TLR7 and/or TLR8 are referred to as TLR7/8 inhibitor. Thus, for example, a TLR7/8 inhibitor may be an inhibitor of TLR7, but not an inhibitor of TLR8. Conversely, a TLR7/8 inhibitor may be an inhibitor of TLR8, but not an inhibitor of TLR7. Preferably, a TLR7/8 inhibitor is both an inhibitor of TLR7 and an inhibitor of TLR8.

TLR7/8 can be inhibited in a variety of ways. Preferably, the TLR7/8 inhibitor is selected from the group consisting of small molecules (low-molecular compounds) and oligonucleotides.

Preferred small molecules are selected from the group consisting of hydroxychloroquine, hydroxychloroquine sulfate, chloroquine, quinacrine (=mepacrine), CpG-52634 (Pfizer (Lipford et al., 2007)), SM934 (Shanghai Institute of Materia Medica (Hou et al., 2011; Wu et al., 2016)), ST2825 (Loiarro et al., 2007; Capolunghi et al., 2010) and combinations thereof. Hydroxychloroquine, hydroxychloroquine sulfate, chloroquine and quinacrine are anti-malaria medicaments with inhibitory effect onto TLR7/8. CpG-52634 is a quinacrine derivative. SM934 is an analog of the anti-malaria medicament artemisinin. ST2825 is a peptidomimetic component which inhibits the MyD88 dimerization of TLRs.

Preferred oligonucleotides are selected from the group consisting of IRS-661 (SEQ ID NO: 21; Dynayax Technologies), IRS-954 (SEQ ID NO: 22; Dynayax Technologies (Barrat et al., 2005, 2007; Guiducci et al., 2010)), DV-1179 (Dynayax Technologies (Zhu et al., 2011; Suarez-Farinas et al., 2013)), IMO-3100 (Idera (Zhu et al., 2011; Suarez-Farinas et al., 2013)), IMO-8400 (Idera (Jiang et al., 2012; Zhu et al., 2012)), IMO-9200 (IderaNivelix), IHN-ODN-24888 (SEQ ID NO: 23; Coley Pharmaceutical GmbH (Rommler at al., 2013, 2015)), ODN 2087 (SEQ ID NO: 24; ODN 2088 Control (ODN2087), MiltenyiBiotec, Bergisch-Gladbach, GER) and combinations thereof. TLR7/8 primarily recognize nucleic acid structures of dsRNA, ssRNA and CpG-DNA. Therefore, oligonucleotides, in particularly the above-mentioned oligonucleotides, can bind to TLR7/8 and act as inhibitors of TLR7/8 by impeding the binding of TLR7/8 to activating ligands and thus inhibiting the activation of TLR7/8.

Other preferred oligonucleotides with inhibitory effect onto TLR7/8 act as indirect inhibitors without any direct physical interaction with TLR7/8, such as described below. Preferably, such indirect inhibitors are in particularly a single stranded RNA which is complementary to miR-574-5p or a single stranded RNA analog which is complementary to miR-574-5p (AntagomiR), in particularly selected from the group consisting of LNA (locked nucleic acid), BNA (bridged nucleic acid), PMO (phosphorodiamidate morpholino oligomer) and PNA (peptide nucleic acid). LNA is particularly preferred. PNA is particularly preferred. Particularly preferably, the inhibitor is a miR-574-5p PNA AntagomiR.

In the sense of the present invention, a TLR7/8 inhibitor can be a direct or an indirect inhibitor.

According to the present invention, a direct TLR7/8 inhibitor is a component which interacts with TLR7/8 in a direct physical manner and thus leads to an inhibition of TLR7/8. Such direct TLR7/8 inhibitors are known to a person skilled in the art and, accordingly, they can be obtained without any problems. Such direct TLR7/8 inhibitors are, for example, described in Gao W., Xiong, Y., Li Q., Yang H. (2017). “Inhibition of Toll-Like Receptor Signaling as a Promising Therapy for Inflammatory Diseases: A Journey from Molecular to Nano Therapeutics.” Frontiers in Physiology, Volume 8, Article 508. However, their use for the reduction of bone resorption has been unknown until now, in particularly in the case of chronic joint diseases. Preferred is a direct TLR7/8 inhibitor of the present invention for use for the reduction of bone resorption in the case of diseases which are selected from the group comprising, preferably consisting of chronic joint diseases, acute joint diseases and periodontitis, and/or in the case of disorders of the engraftment of implants, wherein the inhibitor is selected from the group consisting of hydroxychloroquine, hydroxychloroquine sulfate, chloroquine, quinacrine (=mepacrine), CpG-52634, 5M934, ST2825, IRS-661, IRS-954, DV-1179, IMO-3100, IMO-8400, IMO-9200, IHN-ODN-24888, ODN 2087 (ODN 2088 Control (ODN2087)) and combinations thereof. The invention also relates to a direct TLR7/8 inhibitor of the present invention for use for the reduction of bone resorption in the case of diseases which are selected from the group comprising, preferably consisting of bone metastasis and psoriatic arthritis.

An indirect TLR7/8 inhibitor of the present invention is a component which leads to an inhibition of TLR7/8 by inhibiting one or more TLR7/8 agonists and/or activating one or more TLR7/8 antagonists. An inhibition of TLR7/8 agonists and/or an activation of TLR7/8 antagonists can be achieved by direct physical interaction with the respective TLR7/8 agonists and/or TLR7/8 antagonists. According to the present invention, however, it is also possible that an inhibition of TLR7/8 agonists and/or an activation of TLR7/8 antagonists is achieved by an effect onto respective regulatory elements such as for example promoter and/or enhancer.

Particularly preferred are indirect TLR7/8 inhibitors which inhibit one or more TLR7/8 agonists. TLR7/8 agonists to be inhibited are preferably selected from the group consisting of miR-574-5p, miR-21/29a, let-7 and combinations thereof. Especially preferred indirect TLR7/8 inhibitors are miR-574-5p inhibitors.

Different possibilities for the inhibition of miRs are known to a person skilled in the art and are described, for example, in US 2017/0044541 A1 or US 2009/0286852 A1. In addition, a person skilled in the art knows different antisense methods (for example siRNA). Thus, the provision of a suitable inhibitor against a given miR is possible without any problems.

Preferably, the inhibitor is a single stranded RNA which is completely complementary to miR-574-5p or a single stranded RNA analog which is completely complementary to miR-574-5p (AntagomiR), in particularly selected from the group consisting of LNA (locked nucleic acid), BNA (bridged nucleic acid), PMO (phosphorodiamidate morpholino oligomer) and PNA (peptide nucleic acid). LNA is particularly preferred. PNA is particularly preferred. A preferred sequence of such an inhibitor is shown in SEQ ID NO: 25. The corresponding PNA sequence is shown in SEQ ID NO: 27. Particularly preferably, the inhibitor is a miR-574-5p PNA AntagomiR.

The inhibitor may also be a single stranded RNA which is complementary to miR574-5p or a single stranded RNA analog which is complementary to miR-574-5p (AntagomiR), in particularly selected from the group consisting of LNA (locked nucleic acid), BNA (bridged nucleic acid), PMO (phosphorodiamidate morpholino oligomer) and PNA (peptide nucleic acid), wherein the RNA or the RNA analog is completely complementary to miR-574-5p, but does not cover the whole sequence of miR-574-5p. LNA is particularly preferred. PNA is particularly preferred. The sequence of a particularly preferred inhibitor is shown as RNA sequence in SEQ ID NO: 26 and as PNA sequence in SEQ ID NO: 28. Compared to the sequences SEQ ID NOs: 25 and 27 which cover the whole sequence of miR-574-5p, the sequences SEQ ID NOs: 26 and 28 are shorter by five residues.

Other miR-574-5p inhibitors are also according to the present invention. For example, the invention also comprises so-called “miRNA sponge” and “miRNA decoy”. These are nucleic acid molecules containing tandem binding sites for miR-574-5p. So, they act as competitive inhibitors of miR-574-5p (Ebert and Sharp, 2010 as well as US 2017/0044541 A1). Preferably, the miR-574-5p inhibitor is selected from the group comprising, preferably consisting of AntagomiR, miRNA sponge and miRNA decoy. AntagomiR is particularly preferred. According to the present invention are also miR-574-5p inhibitors which do not directly interact with miR-574-5p, but instead of that have an effect onto regulatory elements of miR-574-5p, for example onto its promoter and/or enhancer.

Preferably, the TLR7/8 inhibitor is selected from the group comprising, preferably consisting of miR-574-5p-AntagomiR, miR574-5p-sponge, miR574-5p-decoy, hydroxychloroquine, hydroxychloroquine sulfate, chloroquine, quinacrine (=mepacrine), CpG-52834, SM934, ST2825, IRS-661, IRS-954, DV-1179, IMO-3100, IMO-8400, IMO-9200, IHN-ODN-24888 and ODN 2087 (ODN 2088 Control (ODN2087)).

According to the present invention, preferably, the extent of the osteoclast differentiation can be used as a measure for the extent of bone resorption. Suitable methods for the ascertainment of the effect of a substance onto the osteoclast differentiation are well-known to a person skilled in the art. Particularly suitable methods are also described in detail in the present section of examples and are also known from literature. In summary, it is advantageous, when the osteoclast differentiation in CD14⁺ monocytes or M2-like macrophages is induced with the help of a TLR7/8 agonist (preferably miR-574-5p) in the presence or absence of the potentially inhibitory substance to be tested each, wherein then the desired inhibitory effect has to be proclaimed, when in the presence of the substance to be tested, in comparison to the absence of the substance, a significantly lower osteoclast differentiation can be observed. Other test methods are also possible. For example, in an alternative or in addition, the extent of the osteoclast differentiation and/or the extent of the bone resorption can be examined in an animal model, in particularly an arthritis mouse model such as CIA (collagen-induced arthritis).

The present invention also relates to a pharmaceutical composition comprising a TLR7/8 inhibitor according to the present invention, in particularly an indirect TLR7/8 inhibitor, particularly preferably a miR-574-5p inhibitor.

Preferably, the pharmaceutical composition contains carriers which are selected from the group consisting of cell penetrating peptides (CPPB), nanocarriers (NCs) and/or cholesterol, or from the group consisting of nanocarriers (NCs) and/or cholesterol. Preferably, the carriers are NCs. Preferred NCs are nanoparticles (NPs). Particularly preferably, the carriers are nanoparticles, in particularly iron oxide nanoparticles, gold nanoparticles and/or silver nanoparticles. Iron oxide nanoparticles, in particularly superparamagnetic iron oxide nanoparticles (SPIONs) are particularly preferred.

Iron oxide nanoparticles are biologically degradable so that there is no excessive undesired accumulation of the particles in the body. A further advantage of iron oxide nanoparticles is that they, also in the case of systemic administration (for example intravenous), can be accumulated by applying an outer magnetic field in the desired body region, in particularly in an affected joint.

Preferably, the nanoparticles of the invention have a diameter in a range of 5 nm to 100 nm, further preferably 7.5 nm to 75 nm, further preferably of 10 nm to 50 nm, for example 15 nm to 40 nm or 20 nm to 30 nm, in particularly about 25 nm. The low diameter of the nanoparticles is particularly advantageous for their free mobility within a membrane. The diameter of the nanoparticles can, for example, be determined by means of dynamic light scattering (DLS) or by means of scanning electron microscopy (SEM), wherein in the case of an SEM determination the diameter is preferably the Martin diameter.

Especially preferably, the pharmaceutical composition contains carriers, wherein the carriers are cell penetrating peptides (CPPs). CPPs are polycationic peptides which are characterized by a plurality of advantages. Optional CPPs are penetratin, TAT (transactivator of transcription), MAP (model amphiphatic peptide), polyarginines (e.g., R3, R4, R5, R6, R7, R8, R9, R10, R11 or R12), pVEC, transportan, MPG, and combinations thereof. A particularly preferred CPP is (N-terminus)-GRKKRWFRRRRMKWKK-(C-terminus) (SEQ ID NO: 29). CPPs allow a simple upscaling, in particularly using solid phase synthesis methods. CPPs involve membrane permeability and nucleolar accumulation.

Preferably, the inhibitor is present in a form conjugated to the carriers. Particularly preferably, the inhibitor is covalently bound to the carrier. Particularly preferably, the inhibitor is a miR-574-5p PNA AntagomiR. Particularly preferable is a miR-574-5p PNA AntagomiR which is covalently bound to CPP, in particularly a miR-574-5p PNA AntagomiR which is covalently bound to CPP by means of a PEG linker. The compounds which are covalently bound to CPP are bioorthogonal. They allow a stronger hybridization and an increased half-life. The PEG linker may comprise one or more ethylene glycol units. In an embodiment, the PEG linker contains one ethylene glycol unit, two or more ethylene glycol units or three or more ethylene glycol units. By a suitable selection of the linker length, it is possible to adjust the solubility.

The TLR7/8 inhibitor, in particularly a miR-574-5p-AntagomiR, can be bound to lysine or a modified lysine. This is in particularly true for PNA-AntagomiR. Particularly preferably, the C-terminal end of a PNA-AntagomiR is bound to a modified lysine, wherein the modification of the lysine preferably consists in the presence of an amide group (—CONH2) instead of the carboxyl group (—COOH). This is advantageous for the synthesis of the PNA by means of solid phase synthesis. In addition, the solubility of the PNA is increased.

Particularly preferably, the inhibitor is densely packed on the carrier. This results in a particularly good free mobility within a membrane. In addition, the stability is increased. Preferably, the surface density of the TLR7/8 inhibitor on the carrier, in particularly of a miR-574-5p inhibitor (preferably AntagomiR) on the nanoparticles, is at least 10¹⁰ molecules per cm², further preferably at least 10¹¹ molecules per cm², further preferably at least 10¹² molecules per cm². However, preferably, the surface density is at most 10¹⁴ molecules per cm², further preferably at most 5*10¹³ molecules per cm², further preferably at most 2*10¹³ molecules per cm². The surface density may, for example, be in a range of 5 to 150, in particularly 10 to 70 AntagomiR oligonucleotides per 10 nm particle.

The present invention also relates to a method for the production of a pharmaceutical composition of the invention, comprising the following steps:

providing a TLR7/8 inhibitor according to the present invention, in particularly an indirect TLR7/8 inhibitor, particularly preferably a miR-574-5p inhibitor,
coupling the TLR7/8 inhibitor to a carrier which is selected from the group consisting of cell penetrating peptides (CPPs), nanocarriers (NCs) and/or cholesterol, or from the group consisting of nanocarriers (NCs) and/or cholesterol, in particularly to NPs, particularly preferably to NPs which are selected from the group consisting of iron oxide nanoparticles, gold nanoparticles and/or silver nanoparticles, especially preferably to iron oxide nanoparticles, or to cell penetrating peptides (CPPs).

Preferably, the coupling according to step b) is achieved via click chemistry, for example such as described in Cutler J. I., Zheng., Xu X., Giljohann D. A., Mirkin C. A. (2010). “Polyvalent Oligonucleotide Iron Oxide Nanoparticle “Click” Conjugates.” Nano Letters 10: 1477-1480. Particularly preferably, carriers, in particularly nanoparticles, which are in aminated form are reacted with azidobutyrate or azidoacetate so that a free azide group is formed. Then, the azide group is used preferably for binding the inhibitor to the carrier. For example, the azide group may form a bond with modified nucleotides, preferably with a stoichiometry of 1:1. As described above, the miR-574-5p inhibitors according to the present invention are preferably RNA or RNA analogs so that these inhibitors can be coupled particularly well via respectively modified nucleotides, in particularly at the 5′ end or at the 3′ end (preferably at the 5′ end), to respectively modified carriers, in particularly nanoparticles, particularly preferably iron oxide nanoparticles. In this connection, a particularly preferable nucleotide modification is a terminal alkyne group, in particularly at the 5′ end of the nucleotide.

The coupling according to step b) to CPPs is preferably achieved by means of a PEG linker. Within the scope of the present invention, it is possible to synthesize CPP and TLR7/8 inhibitor separately and then to couple them subsequently. However, it is preferred to continuously build up the whole construct monomer by monomer. Particularly preferred are embodiments in which the inhibitor is a miR-574-5p PNA AntagomiR. In such embodiments, the CPP-PNA construct is preferably built up starting with the most C-terminal PNA monomer (or the still more C-terminal modified lysine) and ending with the most N-terminal amino acid of the CPP part.

The present invention also relates to a method for the prognosis of the severity of the course of the disease rheumatoid arthritis of a patient comprising the following steps: determining the content of miR-574-5p in sEVs (small extracellular vesicles) in a sample of the patient,

comparing the content determined in step a) with the content of miR-574-5p in sEVs in samples of one or more comparison patients with a known course of the disease, prognosticating the severity of the course of the disease based on the result of the comparison.

The sample of the patient may, for example, be a sample of the synovial fluid or a blood sample, in particularly a plasma sample. Particularly preferably, the sample of the patient is a sample of the synovial fluid.

Preferably, the sEVs are synovial sEVs.

Preferably, the content of miR-574-5p is determined with RT-qPCR.

The present invention also relates to the use of a TLR7/8 inhibitor for the reduction of bone resorption in the case of diseases which are selected from the group comprising, preferably consisting of chronic joint diseases, acute joint diseases and periodontitis, and/or in the case of disorders of the engraftment of implants. Preferred is the use for the reduction of bone resorption in the case of chronic joint diseases. The present invention also relates to the use of a TLR7/8 inhibitor for the reduction of bone resorption in the case of diseases which are selected from the group comprising, preferably consisting of bone metastasis and psoriatic arthritis.

The present invention also relates to a method for the reduction of bone resorption in the case of diseases which are accompanied by an excessive bone resorption, in particularly in the case of diseases which are selected from the group consisting of chronic joint diseases, acute joint diseases and periodontitis, and/or in the case of disorders of the engraftment of implants, wherein the method comprises the step of administering a TLR7/8 inhibitor according to the present invention. The disease is in particularly a chronic joint disease which is selected from the group consisting of rheumatoid arthritis and arthrosis. The diseases may also be or comprise bone metastasis or psoriatic arthritis.

### BRIEF DESCRIPTION OF THE FIGURES

FIG. 1 shows schematically the isolation of sEVs with differential ultracentrifugation.

FIG. 2 shows schematically the induction of osteoclast genesis.

FIG. 3 shows a significant concentration-dependent increase of the osteoclasts, when sEVs are added to monocytes (FIG. 3A) or to M2-like macrophages (FIG. 3B). The results are indicated as mean value+SEM (standard error of the mean). Differences were classified as significant for p<0.05 (indicated as * for p<0.05, ** for p<0.01, *** for p<0.001).

FIG. 4 shows the content of different miRs in the isolated vesicles determined with the help of RT-qPCR. A high content of miR-574-5p (SEQ ID NO: 4) was ascertained in both the sEVs from the serum and the sEVs from the synovial fluid (FIGS. 4A and 4B). The results are indicated as mean value+SEM.

FIG. 5 shows intracellular levels and sEV levels of the indicated miRs with and without stimulation for 24 hours with IL-1β (10 ng/ml) and/or TNFα (10 ng/ml).

FIG. 6 shows intracellular levels (FIG. 6A) and secretion (FIG. 613) of miR-574-5p during the osteoclast differentiation.

FIG. 7A shows the content of miR-574-5p in miR-574-5p oe sEVs and in a control (ScrC sEVs). FIG. 7B shows results of an RNase protection experiment, whereby the RNase degradation of the miR-574-5p can significantly be increased by an addition of a detergent.

FIG. 8 shows the results of a treatment with 1 μg/ml miR-574-5p oe sEVs or ScrC sEVs at different time points of the differentiation. The miR-574-5p oe sEVs lead to a significant increase of the osteoclast number, when added to monocytes or M2-like macrophages (FIGS. 8A and 8B). In the case of the addition to pre-osteoclasts, on the other hand, such a significant increase of the osteoclast number was not observed (FIG. 8C).

FIG. 9 shows that neither miR-574-5p alone, nor miR-574-5p in the presence of synthetic liposomal vehicles (Lipofectamine® 2000) results in a stimulation of the osteoclast genesis.

FIG. 10 shows a direct interaction of miR-574-5p with TLR8 by means of MST (microscale thermophoresis). The constant of dissociation K_{D} was 30.8±5.2 nM (FIGS. 10A and 10B). However, a specific interaction between TLR8 and miR-16-5p was not observed (FIGS. 10A and 10B). FIGS. 10C and 10D show that an addition of the TLR7/8 inhibitor ODN 2087 nullifies the effect of miR-574-5p onto CD14⁺ monocytes and M2-like macrophages. FIGS. 10E and 10F show the influence of the TLR7/8 ligand R848 onto the osteoclast genesis.

FIG. 11 shows the relative IL-23 and IFNα mRNA levels after stimulation of CD14⁺ monocytes with the indicated substances in the presence or absence of the TLR7/8 inhibitor ODN 2087.

FIG. 12 shows schematically the structure of a PNA-AntagomiR (3) coupled to CPP (1). The PNA-AntagomiR (3) is bound to the cell penetrating peptide (1) by means of a PEG linker (2).

FIG. 13 shows the results of an MTT assay for testing the cytotoxicity, wherein on the y-axis the viability is plotted. Neither in the case of the negative control (FIG. 13A) nor in the case of miR-574-5p PNA AntagomiR (FIG. 13B) a significant toxicity was observed.

FIG. 14 shows that the CPP-coupled miR-574-5p PNA AntagomiR results in a concentration-dependent reduction of the osteoclast genesis.

### DETAILED DESCRIPTION OF THE INVENTION

### Examples

Isolation of sEVs from the synovial fluid and the serum of ACPA+RA patients

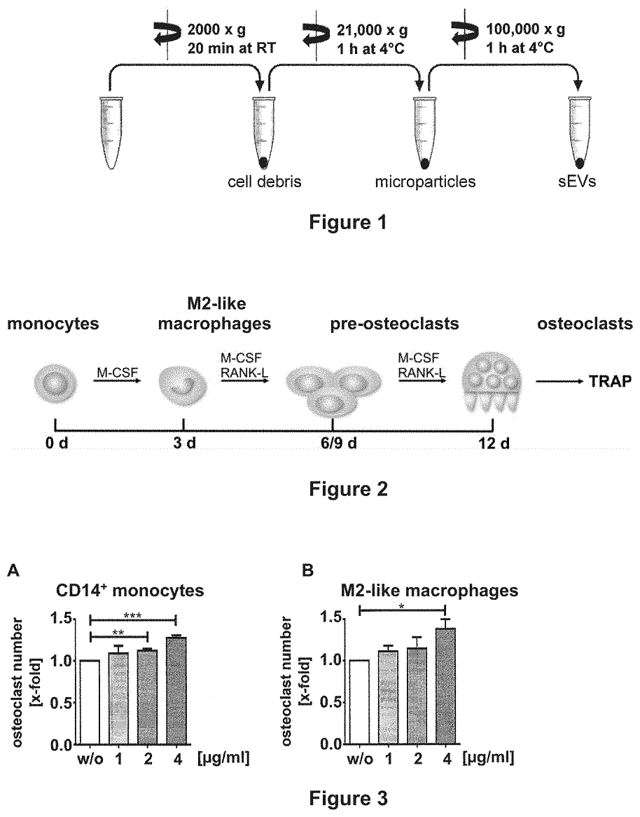

From the synovial fluid of patients with rheumatoid arthritis who were positive for ACPAs (anti-citrullinated protein antibodies) sEVs (small extracellular vesicles, sEVs) were isolated. ACPAs are associated with a more severe course of the disease. The isolation of the vesicles was achieved with differential ultracentrifugation (FIG. 1).

With the help of transmission electron microscopy (TEM) it has been shown that the isolated population was characterized by the typical vesicular morphology and sEVs size of 30 nm to 150 nm. With Western blot, in addition, it has been determined that typical markers of sEVs (CD63, CD9, Hsp70 and CD81) were present. According to this, the isolated vesicles are sEVs.

In the same way, sEVs were also isolated from the serum of the patients.

Induction of osteoclast genesis by the vesicles isolated from the synovial fluid

CD14⁺ monocytes were isolated from PMBCs (peripheral blood mononuclear cells) of healthy donors and stimulated with M-CSF (recombinant human macrophage colony-stimulating factor), RANK-L (receptor activator of NF-κB ligand) and different concentrations of the isolated sEVs. With the sEVs isolated from the synovial fluid both freshly isolated monocytes and differentiated M2-like macrophages were stimulated (FIG. 2).

After 9 to 12 days the cells were fixed and tested for the osteoclast marker TRAP (tartrate-resistant acid phosphatase). TRAP positive cells which also comprised at least three nuclei were classified as osteoclasts and counted with the light microscope.

A significant concentration-dependent increase of the osteoclasts of about 30% was observed, when sEVs were added to monocytes (FIG. 3A). A similar increase was achieved, when the sEVs were added to M2-like macrophages (FIG. 3B). The results are indicated as mean value+SEM (standard error of the mean). The statistical analysis was conducted with an unpaired two-sided t test using the software GraphPad Prism 6.0. Differences were classified as significant for p<0.05 (indicated as * for p<0.05, ** for p<0.01, *** for p<0.001 and *′ for p<0.0001).

The results show that the isolated sEVs induce the osteoclast differentiation in a concentration-dependent manner.

High Content of miR-574-5p in Isolated Vesicles

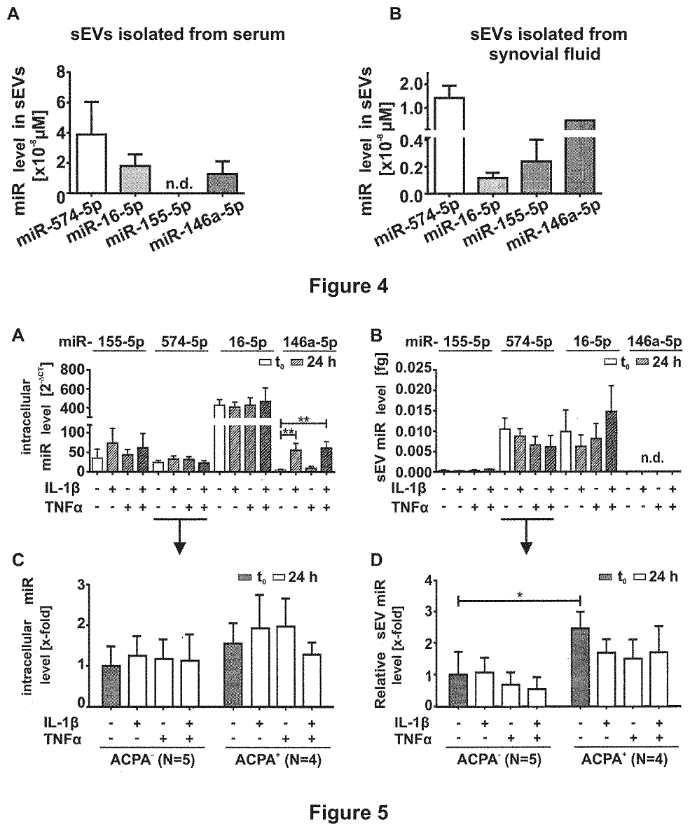

With the help of RT-qPCR the content of different miRs in the isolated vesicles was determined. A high content of miR-574-5p (SEQ ID NO: 4) was determined in both the sEVs from the serum and the sEVs from the synovial fluid (FIGS. 4A and 4B). The results are indicated as mean value+SEM. On the other hand, the content of the other miRs tested (miR-16-5p, miR-155-5p and miR-146a-5p; SEQ ID NOs: 1-3) was considerably lower. The primers were purchased from the company Qiagen (Hilden, Germany) each (catalog number MS00043617 for miR-574; M50031493 for miR-16; MS00031486 for miR-155; and MS00003535 for miR-146a).

As a control for the normalization of the extracellular content non-human cel-miR39-3p (SEQ ID NO: 5) was used, which was added to the samples for this purpose in an end concentration of 200 nM. The primers also came from Qiagen (catalog number MS00019789).

The results show a selective accumulation of miR-574-5p in sEVs of patients with rheumatoid arthritis.

Synovial fibroblasts and monocytes as cellular sources of extracellular miR-574-5p

Synovial fibroblasts (SFs) were obtained from ACPA negative and ACPA positive patients with rheumatoid arthritis. Intracellular levels and sEV levels of the above-mentioned miRs were determined with RT-qPCR with and without stimulation for 24 hours with IL-1β(10 ng/ml) and/or TNFα (10 ng/ml) (FIGS. 5A and 5B). The sEVs were isolated from the supernatants of the cell cultures. For the normalization of the extracellular content always cel-miR-39-3p was used. For the normalization of the intracellular content always snRNA U6 (SEQ ID NO: 20) served as an endogenous control. The primers also came from Qiagen (catalog number M500033740).

miR-146a-5p served as a positive control, because its induction by stimulation with IL-1β is known (Stanczyk J., Pedrioli D. M. L., Brentano F., Sanchez-pernaute Q., Kolling C., Gay R. E., Detmar M., Gay S. and Kyburz D. (2008). “Altered Expression of MicroRNA in Synovial Fibroblasts and Synovial Tissue in Rheumatoid Arthritis.” Arthritis Rheum 58: 1001-1009). For the other miRs no significant stimulation-related differences could be observed.

Noticeable is the selective accumulation of miR-574-5p in the sEVs. While the intracellular content of miR-574-5p in the SFs in comparison to the intracellular content of miR-16-5p is considerably lower, in the isolated sEVs comparable contents of both miRs can be found (FIGS. 5A and 5B).

Interestingly, in addition, differences between SFs from ACPA negative (ACPA⁻) and ACPA positive (ACPA⁺) patients were observed. ACPAs are associated with a more severe course of the disease, and insofar they are suitable as an indicator of the severity of the course of the disease. As shown in FIG. 5D, in sEVs of SFs from ACPA positive patients in comparison to ACPA negative patients significantly increased amounts of miR574-5p were determined so that a connection can be established between the content of extracellular miR-574-5p and the severity of the course of the disease.

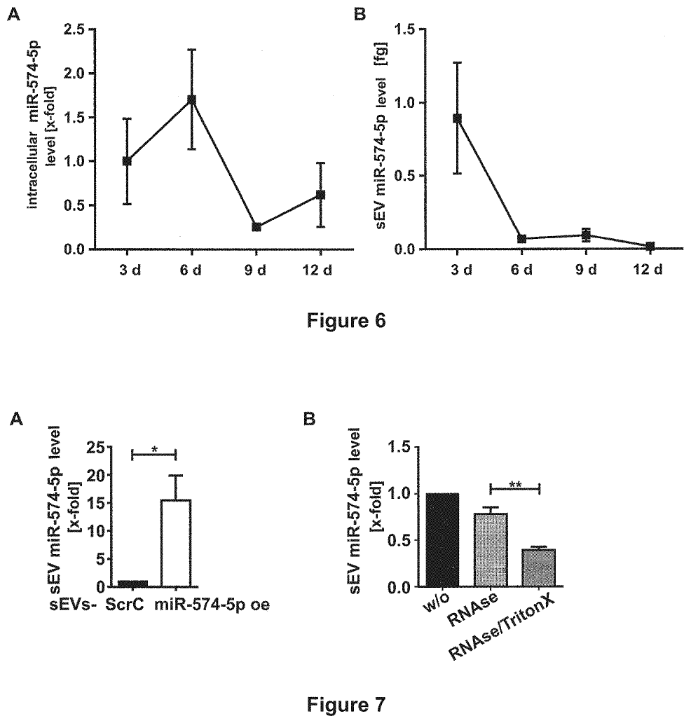

Besides SFs also M2 macrophages secrete miR-574-5p in sEVs (FIG. 6). While the intracellular level during the osteoclast differentiation substantially remains unchanged (FIG. 6A), a substantial secretion after 6, 9 or 12 days of differentiation does not take place any longer (FIG. 6B). Thus, pre-osteoclasts and osteoclasts do not substantially contribute to miR-574-5p in sEVs.

In summary, SFs and monocytes, however not pre-osteoclasts and osteoclasts contribute to miR-574-5p in sEVs.

sEVs with Designed miR-574-5p Level

With the help of XMIRXpress constructs (System Bioscience, Palo Alto, USA) in HEK 293 cells sEVs with different contents of miR-574-5p were designed. The content of miR-574-5p in the miR-574-5p oe sEVs was about 15 times higher in comparison to the control (ScrC sEVs) which was obtained with the help of a control miR (XMIRXP-NT, System Biosciences, Palo Alto, USA) (FIG. 7A).

In an RNase protection experiment it has been shown that the RNase degradation of the miR-574-5p can significantly be increased by an addition of a detergent (FIG. 7B). Thus, miR-574-5p is located in the vesicles which protect against RNase degradation, and it only becomes accessible for the degradation by RNase, when released by the detergent. This experiment shows that the miR-574-5p is mainly located in the sEVs and thus is protected against degradation by RNases. Only through the addition of the detergent a degradation of the miR is possible by the “destruction” of the sEVs.

Uptake of the Designed sEVs by Cells

miR-574-5p oe sEVs were stained with the fluorescent dye 3,3′-dioctadecyloxacarbocyanine perchlorate (DiO), and their uptake in CD14⁺ monocytes was examined by confocal microscopy. After 20 minutes an uptake of the stained vesicles in the cells was observed. The maximum number of stained sEVs in the cells was achieved after 40 minutes. Similar results were obtained for HeLa cells.

Induction of the Osteoclast Genesis by sEVs with High Content of miR-574-5p

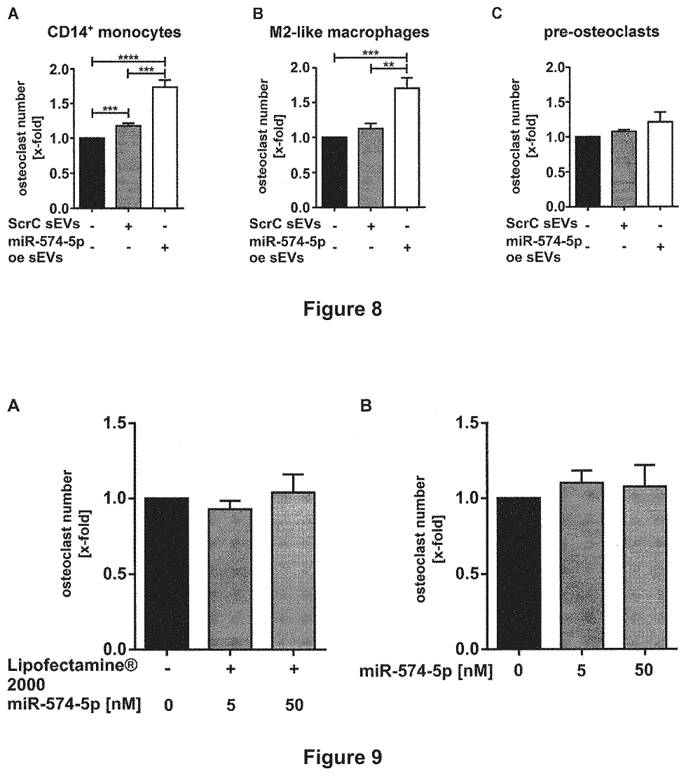

Isolated human CD14⁺ monocytes were treated with 1 μg/ml miR-574-5p oe sEVs or ScrC sEVs at different time points of the differentiation (FIG. 8A, 8B, 8C).

The miR-574-5p oe sEVs result in a significant increase of the number of osteoclasts, when added to monocytes or M2-like macrophages (FIGS. 8A and 8B). Whereas in the case of an addition to pre-osteoclasts no significant increase of the number of osteoclasts was observed (FIG. 8C).

The ScrC sEVs also result in a significant increase of the number of osteoclasts, when added to monocytes or M2-like macrophages (FIGS. 8A and 8B). This is probably due to the miR-574-5p which is also present there, but the content of which in comparison to the miR-574-5p oe sEVs is lower by a factor of 15 (FIG. 7A) so that the stimulation of the osteoclast genesis with about 1.2-fold in the case of ScrC sEVs is considerably lower than in the case of miR-574-5p oe sEVs with about 1.7-fold.

The observed stimulation effect depends on the presence of miR-574-5p in sEVs. Neither miR-574-5p alone, nor miR-574-5p in the presence of synthetic liposomal vehicles (Lipofectamine® 2000 (Thermo Fisher Scientific, Waltham, USA)) results in a stimulation of the osteoclast genesis (FIG. 9).

Induction of the Osteoclast Genesis Mediated by Interaction of miR-574-5p with TLR7/8

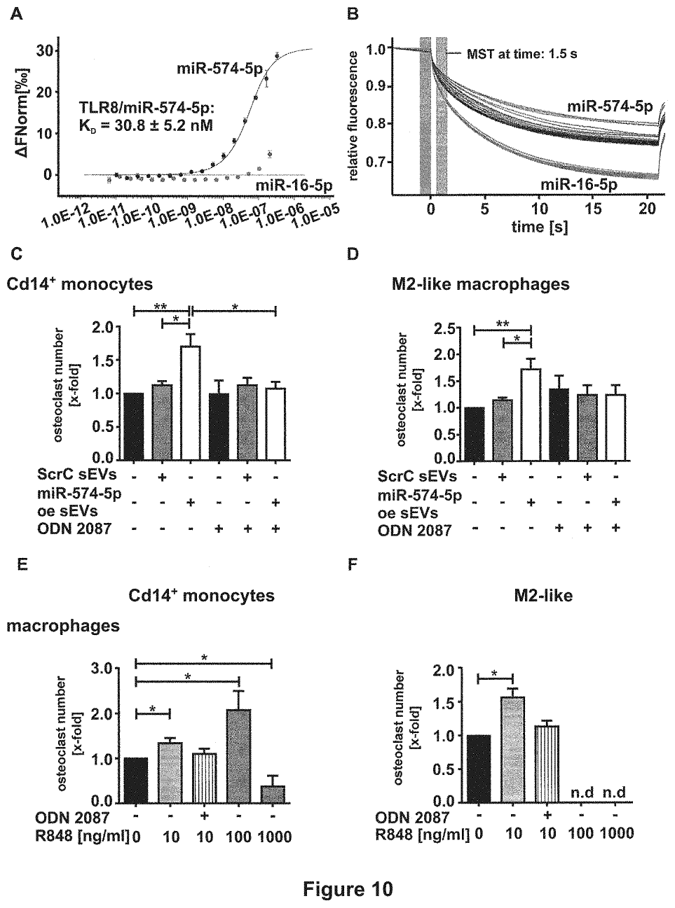

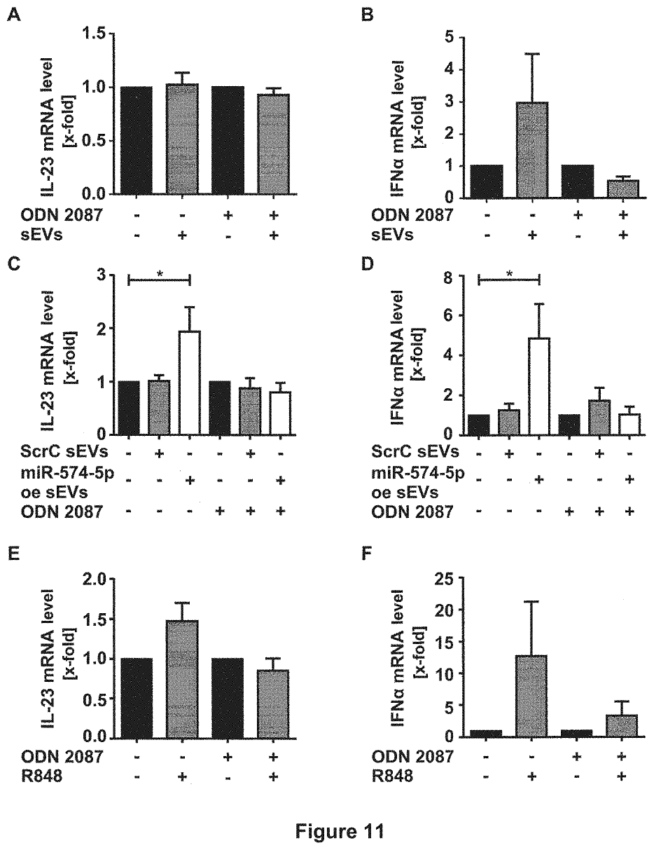

In the context of the present invention, a direct interaction of miR-574-5p with TLR8 was detected by means of MST (microscale thermophoresis); (Wienken C. J., Baaske P., Rothbauer U., Braun D. and Duhr S. (2010). “Protein-binding assays in biological liquids using microscale thermophoresis.” Nat Commun 1: 100). The constant of dissociation K_{D} was 30.8±5.2 nM (FIGS. 10A and 10B). However, a specific interaction between TLR8 and miR-16-5p was not shown (FIGS. 10A and 10B). For the MST experiments Cy5-labeled miR-574-5p (SEQ ID NO: 18) and Cy5-labeled miR-16-5p (SEQ ID NO: 19) were used.

An addition of the TLR7/8 inhibitor ODN 2087 (ODN 2088 Control (ODN2087), MiltenyiBiotec, Bergisch-Gladbach, GER) nullifies the above-described effect of miR-574-5p onto CD14⁺ monocytes and M2-like macrophages so that miR-574-5p in the presence of ODN 2087 can not result in a significant increase of the number of osteoclasts (FIGS. 10C and 10D).

Conversely, an increase of the osteoclast genesis can be achieved by an addition of the known TLR7/8 ligand R848 (Invivogen, San Diego, USA). But the effect of R848 strongly depends on the concentration. While in the case of 10 ng/ml a significant increase of the number of osteoclasts can be observed, a reduction of the number of osteoclasts arises in the case of 1000 ng/ml. 100 ng/ml of R848, when added to monocytes, result in an increase of the number of osteoclasts, whereas, when added to M2-like macrophages, in a decrease of the same (FIGS. 10E and 10F). It was possible to nullify the effect by an addition of the TLR7/8 inhibitor ODN 2087. As with miR-574-5p (FIG. 8C), no significant increase of the osteoclast genesis was observed, when the TLR7/8 agonist R848 was added to pre-osteoclasts.

The results shown in FIG. 10 verify that the effect of miR-574-5p oe sEVs onto the increase of the number of osteoclasts is mediated by an interaction with TLR7/8.

miR-574-5p Induces IFNα and IL-23 mRNA in CD14⁺ Monocytes by TLR7/8 Activation

CD14⁺ monocytes were stimulated with sEVs from the synovial fluid of ACPA positive patients with rheumatoid arthritis (4 μg/ml), with miR-574-5p oe sEVs (1 μg/ml) or with ScrC sEVs (1 μg/ml) for 4 hours each. Then the whole RNA was isolated and the mRNA levels of IL-23, IL-8, INFα, IL-1β and TNFα were analyzed with RT-qPCR. These cytokines are known for their influence onto the differentiation of osteoclasts (Amara-sekara D. S., Yun Ii., Kim S., Lee N. and Rho J. (2018). “Regulation of Osteoclast Differentiation by Cytokine Networks.” Immune Netw 18:
1-18). The sequences of the primer pairs used are shown in the sequence protocol (SEQ ID NOs: 6-7 for INFα, SEQ ID NOs: 8-9 for IL-23, SEQ ID NOs: 10-11 for TNFα, SEQ ID NOs:
   12-13 for IL-1β and SEQ ID NOs: 14-15 for IL-8). For the normalization of the cDNA amounts in different samples the levels of β-actin were used (primer pair: SEQ ID NOs: 16-17).

The levels of TNFα, IL-1β and IL-8 were not influenced by miR-574-5p under any of the conditions tested. On the contrary, with a stimulation with sEVs which were obtained from the synovial fluid of patients with rheumatoid arthritis an about three-fold increase of the INFα mRNA could be observed (FIG. 118). Stimulation with miR-574-5p oe sEVs even resulted in an about five-fold increase of the INFα mRNA and in an about two-fold increase of the IL-23 mRNA, while with ScrC sEVs no increase was observed (FIGS. 11C and 11D). Comparable results were achieved by stimulation of the monocytes with 10 ng/ml of the TLR7/8 ligand R848 (FIGS. 11E and 11F). It was possible to nullify the observed effects by an addition of the TLR7/8 inhibitor ODN 2087.

Reduction of the Osteoclast Genesis by miR-574-5p AntagomiR Coupled to CPP

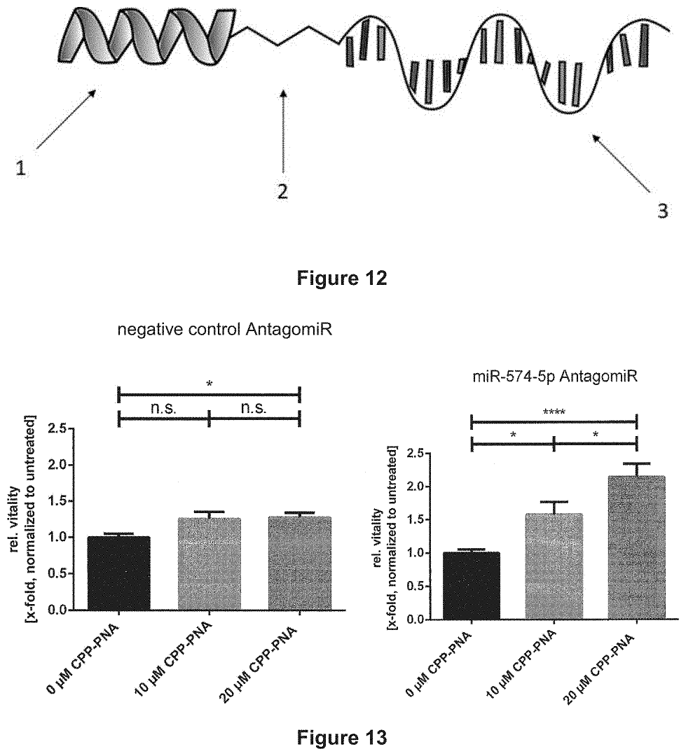

A miR-574-5p PNA AntagomiR was covalently bound via a PEG linker to a cell penetrating peptide (CPP) (see scheme in FIG. 12). The miR-574-5p PNA AntagomiR is a single stranded RNA analog which is complementary to miR-574-5p, namely a so-called “peptide nucleic acid” (PNA). The AntagomiR is completely complementary to miR574-5p, but it does not cover the whole sequence of miR-574-5p. The sequence of AntagomiR is shown in SEQ ID NO: 28. At the C-terminus the AntagomiR sequence was connected with a modified lysine. The modification consisted of the presence of an amide group (—CONH2) instead of the carboxyl group (—COOH). In the synthesis, the modified lysine is used for anchoring to the resin scaffold (see next paragraph). As a spacer with respect to the resin the modified lysine improves the efficiency of the first critical coupling. Furthermore, the modified lysine increases the solubility of the CPP-PNA construct.

Standard methods of solid phase synthesis were used. The whole CPP-PNA construct was built up on a resin scaffold, because all the synthesis building blocks used are compatible with each other. The synthesis was conducted from the C-terminus to the N-terminus. At first, the most C-terminal synthesis building block (here modified lysine) was immobilized on a resin scaffold. The next more N-terminal synthesis building block was then reacted with the synthesis building block which has been immobilized on the resin. This reaction cycle was repeated for each building block so that the macromolecule located on the resin grew building block by building block. When the most N-terminal building block has been reacted, then the finished CPP-PNA construct was released from the resin scaffold.

The CPP-PNA construct used can be illustrated as follows (from the N-terminus to the C-terminus): GRKKRWFRRRRMKWKK-(eg1)-ctcacacacacacactca(K—CONH2). The first part is the CPP (SEQ ID NO: 29). The expression (eg-1) describes the PEG linker which here comprises exactly one ethylene glycol unit. Then the PNA AntagomiR with SEQ ID NO: 28 follows. Terminatory, the construct contains a modified lysine with an amide group (—CONH2) instead of a carboxyl group (—COOH). The modified lysine is described by the expression (K—CONH2).

The cytotoxicity was tested with an MTT assay in which the eponymous dye MTT (3-(4,5-dimethylthiazol-2-yl)-2,5-diphenyltetrazoliumbromide) is used. As negative control a PNA with a sequence which was not complementary to miR-574-5p and which was also bound to CPP by means of the PEG linker was tested (SEQ ID NO: 30). The CPP-PNA construct of the negative control used can be illustrated as follows (from the N-terminus to the C-terminus): GRKKRWFRRRRMKWKK-(eg1)-gctattaccttaacccag(K—NH2). The negative control differs from the above-described construct according to the present invention only with respect to the PNA sequence.

The results are shown in FIG. 13, wherein on the y-axis the viability is plotted. Neither in the case of the negative control (FIG. 13A) nor in the case of miR-574-5p PNA AntagomiR (FIG. 13B) a significant toxicity was observed. In the case of miR-574-5p PNA AntagomiR even an increased viability in comparison to the untreated control was observed. The cytotoxicity was tested on freshly isolated human monocytes.

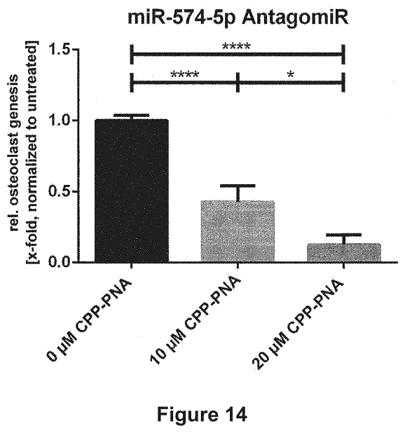

The influence of the miR-574-5p PNA AntagomiR which was coupled to CPP onto the osteoclast genesis was tested. The scheme of the induction of the osteoclast genesis is shown in FIG. 2. This scheme has been adapted such that on day 1 the CPP-coupled miR-574-5p PNA AntagomiR (CPP-PNA) was added in concentrations of 10 μM or 20 μM. The negative control was conducted without any addition of CPP-PNA (0 μm CPP-PNA). The results are shown in FIG. 14. The CPP-coupled miR-574-5p PNA AntagomiR results in a concentration-dependent reduction of the osteoclast genesis. Also for this experiment freshly isolated human monocytes were used. After the completion of the differentiation matured osteoclasts were visualized with the help of histologic staining (detection of tartrate-resistant acid phosphatases, TRAP). Subsequently, multinuclear TRAP positive cells are quantified under a light microscope.

### LIST OF REFERENCE SIGNS

1 cell penetrating peptide (CPP)
2 PEG linker
3 miR-574-5p PNA AntagomiR

What is claimed is:

1. A method comprising the step of administering a TLR7/8 inhibitor for the reduction of bone resorption in the case of diseases which are accompanied by an excessive bone resorption, in particularly in the case of diseases which are selected from the group consisting of chronic joint diseases, acute joint diseases and periodontitis, and/or in the case of disorders of the engraftment of implants.

2. The method according to claim 1, wherein the disease is a chronic joint disease which is selected from the group consisting of rheumatoid arthritis and arthrosis.

3. The method according to claim 1, wherein the TLR7/8 inhibitor is a direct or an indirect inhibitor, wherein a direct TLR7/8 inhibitor is a component which interacts with TLR7/8 in a direct physical manner and thus results in an inhibition of TLR7/8, and wherein an indirect TLR7/8 inhibitor is a component which results in an inhibition of TLR7/8 by inhibiting one r more TLR7/8 agonists and/or activating one or more TLR7/8 antagonists.

4. The method according to claim 1, wherein the inhibitor is selected from the group consisting of miR-574-5p-AntagomiR, miR574-5p-sponge, miR574-5p-decoy, hydroxychloroquine, hydroxychloroquine sulfate, chloroquine, quinacrine (=mepacrine), CpG-52634, SM934, ST2825, IRS-661, IRS-954, DV-1179, IMO-3100, IMO-8400, IMO-9200, IHN-ODN-24888 and ODN 2087 (ODN 2088 Control (ODN2087)).

5. The method according to claim 1, wherein the inhibitor is a single stranded RNA analog which is complementary to miR-574-5p (SEQ ID NO: 4) (AntagomiR).

6. The method according to claim 5, wherein the inhibitor is a PNA AntagomiR.

7. The method according to claim 1, wherein the bone resorption is reduced by means of an inhibition of the osteoclast differentiation.

8. A pharmaceutical composition comprising a TLR7/8 inhibitor according to claim 1.

9. The pharmaceutical composition according to claim 8, wherein the composition contains carriers which are selected from the group consisting of nanocarriers (NCs) and/or cholesterol.

10. The pharmaceutical composition according to claim 9, wherein the NCs are nanoparticles (NPs).

11. The pharmaceutical composition according to claim 9, wherein the carriers are iron oxide nanoparticles.

12. The pharmaceutical composition according to claim 10, wherein the nanoparticles have a diameter in a range of 5 nm to 100 nm.

13. The pharmaceutical composition according to claim 9, wherein the inhibitor is present covalently bound to the carrier.

14. The pharmaceutical composition according to claim 8, wherein the composition contains carriers which are cell penetrating peptides (CPPB).

15. The pharmaceutical composition according to claim 14, wherein the TLR7/8 inhibitor is covalently bound to the CPP by means of a PEG linker.

16. The pharmaceutical composition according to claim 14, wherein the TLR7/8 inhibitor is a single stranded RNA analog which is complementary to miR-574-5p (SEQ ID NO: 4) (AntagomiR).

17. A method for the prognosis of the severity of the course of the disease rheumatoid arthritis of a patient comprising the following steps:
a) determining the content of miR-574-5p in sEVs (small extracellular vesicles) in a sample of the patient,
b) comparing the content determined in step a) with the content of miR-574-5p in sEVs in samples of one or more comparison patients with a known course of the disease, and
c) prognosticating the severity of the course of the disease based on the result of the comparison.

18. The method according to claim 17, wherein the content of miR-574-5p is determined with RT-qPCR.


SEQUENCE LISTING (30 sequences)

SEQ ID NO: 1 - RNA, 22 nt, Homo sapiens
uagcagcacguaaauauuggcg

SEQ ID NO: 2 - RNA, 23 nt, Homo sapiens
uuaaugcuaaucgugauaggggu

SEQ ID NO: 3 - RNA, 22 nt, Homo sapiens
ugagaacugaauuccauggguu

SEQ ID NO: 4 - RNA, 23 nt, Homo sapiens
ugagugugugugugugagugugu

SEQ ID NO: 5 - RNA, 21 nt, Caenorhabditis elegans
caccggguguaaaucagcuug

SEQ ID NO: 6 - DNA, 20 bp, Artificial Sequence
  Primer
gcaagcccagaagtatctgc

SEQ ID NO: 7 - DNA, 20 bp, Artificial Sequence
  Primer
cttgacttgcagctgagcac

SEQ ID NO: 8 - DNA, 20 bp, Artificial Sequence
  Primer
gttccccatatccagtgtgg

SEQ ID NO: 9 - DNA, 20 bp, Artificial Sequence
  Primer
aaaaatcagaccctggtgga

SEQ ID NO: 10 - DNA, 21 bp, Artificial Sequence
  Primer
cccagggacctctctctaatc

SEQ ID NO: 11 - DNA, 21 bp, Artificial Sequence
  Primer
atgggctacaggcttgtcact

SEQ ID NO: 12 - DNA, 21 bp, Artificial Sequence
  Primer
acagatgaagtgctccttcca

SEQ ID NO: 13 - DNA, 21 bp, Artificial Sequence
  Primer
gtcggagattcgtagctggat

SEQ ID NO: 14 - DNA, 20 bp, Artificial Sequence
  Primer
agctctgtgtgaaggtgcag

SEQ ID NO: 15 - DNA, 20 bp, Artificial Sequence
  Primer
tggggtggaaaggtttggag

SEQ ID NO: 16 - DNA, 18 bp, Artificial Sequence
  Primer
cgggacctgactgactac

SEQ ID NO: 17 - DNA, 22 bp, Artificial Sequence
  Primer
cttctccttaatgtcacgcacg

SEQ ID NO: 18 - RNA, 23 nt, Artificial Sequence
  Cyanine5-labeled hsa-miR-574-5p
  modified_base (23)..(23): Cyanine5-labeled uracil
ugagugugugugugugagugugu

SEQ ID NO: 19 - RNA, 22 nt, Artificial Sequence
  Cyanine5-labeled hsa-miR-16
  modified_base (22)..(22): Cyanine5-labeled guanine
uagcagcacguaaauauuggcg

SEQ ID NO: 20 - RNA, 107 nt, Homo sapiens
gugcucgcuucggcagcacauauacuaaaauuggaacgauacagagaagauuagcauggccccugcgcaaggaugacacgcaaauucgugaagcguuccauauuuuu

SEQ ID NO: 21 - DNA, 20 bp, Artificial Sequence
  TLR7/8 inhibitor
tgcttgcaagcttgcaagca

SEQ ID NO: 22 - DNA, 18 bp, Artificial Sequence
  TLR7/8 inhibitor
tgctcctggaggggttgt

SEQ ID NO: 23 - DNA, 15 bp, Artificial Sequence
  TLR7/8 inhibitor
  misc_feature (1)..(15): Phosphorothioate backbone
  modified_base (8)..(8): n is 7-deaza-2′-O-methyl-guanine
tcctggcngggaagt

SEQ ID NO: 24 - DNA, 15 bp, Artificial Sequence
  TLR7/8 inhibitor
  misc_feature (1)..(15): Phosphorothioate backbone
tcctgagcttgaagt

SEQ ID NO: 25 - RNA, 23 nt, Artificial Sequence
  AntagomiR
acacacucacacacacacacuca

SEQ ID NO: 26 - RNA, 18 nt, Artificial Sequence
  AntagomiR
cucacacacacacacuca

SEQ ID NO: 27 - DNA, 23 bp, Artificial Sequence
  Peptide Nucleic Acid (PNA) AntagomiR
acacactcacacacacacactca

SEQ ID NO: 28 - DNA, 18 bp, Artificial Sequence
  Peptide Nucleic Acid (PNA) AntagomiR
ctcacacacacacactca

SEQ ID NO: 29 - protein, 16 aa, Artificial Sequence
  Cell Penetrating Peptide (CPP)
GRKKRWFRRRRMKWKK

SEQ ID NO: 30 - DNA, 18 bp, Artificial Sequence
  Peptide Nucleic Acid (PNA) Negativkontrolle
gctattaccttaacccag